(19) United States
(12) Patent Application Publication
(10) Pub. No.: US 2022/0068454 A1
(43) Pub. Date: Mar. 3, 2022

(54) SYSTEM AND METHOD FOR MEDICAL RESEARCH DATA PROCESSING, ACQUISITION AND ANALYSIS

(71) Applicant: RCR BioPharma, Los Angeles, CA (US)

(72) Inventor: IRENA RASKIN, LOS ANGELES, CA (US)

(73) Assignee: RCR BioPharma, Los Angeles, CA (US)

(21) Appl. No.: 17/463,699

(22) Filed: Sep. 1, 2021

Related U.S. Application Data
(60) Provisional application No. 63/073,098, filed on Sep. 1, 2020.

(51) Int. Cl. G16H 20/10; G16H 10/60; G16H 50/20

(52) U.S. Cl. CPC G16H 20/10; G16H 50/20; G16H 10/60

(57) ABSTRACT

Described is a system and method for medical research data processing, acquisition and analysis. The system employs a master knowledge generator that is operable for receiving, from a private database, patient specific data. Medicine information is received from an integrated knowledge base. A right ratio personalized medicine recommendation is then generated for the patient based on the patient specific data and the medicine information from the integrated knowledge base. Tracker data is received on the patient while the patient is taking the personalized medicine recommendation, Further, the right ratio personalized medicine recommendation is updated for the patient based on the tracker data.

### CROSS-REFERENCE TO RELATED APPLICATIONS

This is a non-provisional application of U.S. Provisional Application No. 63/073,098, filed on Sep. 1, 2020, the entirety of which is incorporated herein by reference.

### BACKGROUND OF INVENTION

### (1) Field of Invention

The present invention relates to medical research and, more specifically, to a system and method for medical research data processing, acquisition and analysis.

### (2) Description of Related Art

The present disclosure is generally directed to medical research data processing, acquisition and analysis. Over centuries mankind has gathered massive volumes of data with the intention of understanding medical, health and wellness more accurately and in the present day data collects itself naturally and is estimated that the amount of medical information is expected to double every 73-days. While the data increases in size so does the need for computer-assisted algorithms (such as Artificial Intelligence) to synthesize, optimize and adapt our ability to improve diagnosis of disease and improve the effectiveness and the way therapies are brought to market.

Thus, a continuing need exists for an improved system that can efficiently process mass volumes of medical research data through efficient acquisition and analysis.

### SUMMARY OF INVENTION

Public health is connected to cannabis with regard to food, animal feed (feed), and pharmaceuticals. Therefore, the use of phytocannabinoids should be examined from a medical research perspective. Currently, knowledge on medical cannabis treatment does not address sufficiently diseases which are of epidemiological and of zoonotic concern. To answer the growing need of scientific evidence-based applicable medicine in both human and veterinary medicine, a new approach for the investigation of the therapeutic potential of cannabinoids must be adopted. A model that offers direct study of a specific disease in human and veterinary patients may facilitate development of novel therapies.

The term animal/murine model is commonly used in medical studies. The Scientific paradigm of pre-clinical trials has not changed throughout the years. Preclinical trials using rodent models are limited in their ability to predict outcomes in human patients. Many medications developed based on rodent models fail to demonstrate clinical efficacy in humans; hence these models are neither time- nor cost-effective. There are numerous reasons why laboratory animal models fail to model human reactions to drugs properly. As opposed to in human and pets, disease does not occur naturally in animal models, and therefore the disease is not the same in the test subjects as in humans. Environmental risk factors for disease occurrence (and environmental factors influencing patients at home) are not comparable between animal models in a lab and human patients at home or in a hospital. This invention recognizes that both human and animal health are connected, together with a ecosystem and from this perspective a better medical research model for drug testing might be found in animals with naturally occurring diseases, treated as a part of their life routine, in the same environment as humans, allowing for the simultaneous development of drugs for human and animal patients, thereby reducing time to development of drugs for humans, reducing associated costs, and contributing to animal welfare.

Companion animals, including dogs, offer a unique opportunity to study the full range of complex factors—both environmental using real world evidence and biological evidence. One of the major challenges in studying complex traits is the need for very large sample sizes, with detailed information on each individual and its environment. Ideally, such information should be collected longitudinally over the lifetime of the individual. Dogs are ideally suited for such research. Most of the tens of millions of dogs in the U.S. and other high-income nations have a human family that monitors their health and wellbeing and will care for them during their life and to old age. As such, the human-dog family become de facto study teams of research and subject that can both enrich the knowledge to the scientific community and enhance the participation by the human community in science.

The present invention relates to medical research and translational medicine and, more specifically, to a system for medical research data processing, acquisition and analysis. The system and method include any or all the computing infrastructure that creates the collection of hardware and software elements including computing power, networking, and storage, as well as an interface for users to access their virtualized resources, services and management tools, that support the computing requirements as may be necessary to implement the system and method as described herein.

In one aspect, the system includes a master knowledge generator that is operable for performing several operations, such as receiving, from a private database, patient specific data for a patient; receiving medicine information from an integrated knowledge base; generating a right ratio personalized medicine recommendation for the patient based on the patient specific data and the medicine information from the integrated knowledge base; receiving tracker data on the patient while the patient is taking the personalized medicine recommendation; and updating the right ratio personalized medicine recommendation for the patient based on the tracker data. It should be noted that the master knowledge generator and other components can be implemented with one or more processors and a memory having instructions encoded thereon, such that when executed, the instructions cause the one or more processors (and associated modules, etc.) to perform the operations listed herein.

In another aspect, the master knowledge generator checks the personalized medicine recommendation against a regulatory database to ensure regulatory compliance.

In yet another aspect, the system includes a computer-assisted acquisition (CAA) module. The CAA module is operable for performing several operations, such as receiving parameters detailing a resource type used to acquire data; receiving parameters detailing a data type and condition of data to acquire; receiving medical information from a device to capture the data; transferring the data that has been captured to a processing module that allocates the data to a computer program for analysis; reporting on the data obtained using a report module; and archiving the data using an archive module.

In another aspect, the system includes a computer-assisted processing (CAP) module. The CAP module is operable for performing several operations of, such as receiving parameters detailing computer resources needed to access additional data (e.g., receiving instructions to locate and access an external database for the purposes of identifying and recording if a patient has had a vaccination or not); receiving parameters detailing a source and data type to acquire the additional data (e.g., receiving instructions to locate and access information that is alphanumeric and is from a Center for Disease Control and Prevention (CDC) approved database); receiving parameters detailing conditions to acquire the additional data (e.g., receiving instructions to locate and obtain the data if the information is missing); updating a patient data record in the private database with the additional data; transferring the additional data to other computer processing modules for analysis and storage; reporting on the data using a report module; and archiving the data using the archive module.

In another aspect, the system includes a computer-assisted data operating algorithms (CAD) module. The CAD module is operable for performing operations, such as receiving parameters, from a computer-assisted program, detailing the data, an analysis of the data, a type of the data, a source of the data and logic needed to conduct artificial intelligence routines that extract features from the data and augment decision making for medical purposes; receiving parameters detailing the conditions to initiate the artificial intelligence/deep learning routines (e.g., such as when CAD 24 receives data from CAA 22, CAD 24 should re-compute the analysis and re-distribute the results to the Private Database 9); updating a patient data record with the logic needed to conduct artificial intelligence/deep learning routines; updating one or more other databases with artificial intelligence/deep learning routines; updating the artificial intelligence/deep learning routines to other computer processing modules for additional analysis and storage; reporting on the additional analysis and artificial intelligence/deep learning using a report module; and archiving the analysis and artificial intelligence/deep learning.

Finally, the present invention also includes a computer program product and a computer implemented method. The computer program product includes computer-readable instructions stored on a non-transitory computer-readable medium that are executable by a computer having one or more processors, such that upon execution of the instructions, the one or more processors perform the operations listed herein. Alternatively, the computer implemented method includes an act of causing one or more processors to execute such instructions and perform the resulting operations.

### BRIEF DESCRIPTION OF THE DRAWINGS

The objects, features and advantages of the present invention will be apparent from the following detailed descriptions of the various aspects of the invention in conjunction with reference to the following drawings, where:

FIG. 1. is a schematic diagram illustrating a data method and system that interfaces with consumers, patients, and clinicians and researchers in accordance with an embodiment of the present invention;

FIG. 2. is a schematic diagram illustrating how various data resources interface with other public and private databases creating an Integrated Knowledge Base used by clinicians, researchers in accordance with an embodiment of the present invention;

FIG. 3 is a schematic diagram illustrating database resources as information repositories which include means for acquiring medical research data through automated, semi-automated, or manual techniques in the Master Knowledge Generator;

FIG. 4 is a table depicting various types of controllable resources;

FIG. 5 is a block diagram depicting the components of a system according to various embodiments of the present invention; and

FIG. 6 is an illustration of a computer program product embodying an aspect of the present invention.

### DETAILED DESCRIPTION

The present invention relates to medical research and, more specifically, to a system for medical research data processing, acquisition and analysis. The following description is presented to enable one of ordinary skill in the art to make and use the invention and to incorporate it in the context of particular applications. Various modifications, as well as a variety of uses in different applications, will be readily apparent to those skilled in the art, and the general principles defined herein may be applied to a wide range of aspects. Thus, the present invention is not intended to be limited to the aspects presented, but is to be accorded the widest scope consistent with the principles and novel features disclosed herein.

In the following detailed description, numerous specific details are set forth in order to provide a more thorough understanding of the present invention. However, it will be apparent to one skilled in the art that the present invention may be practiced without necessarily being limited to these specific details. In other instances, well-known structures and devices are shown in block diagram form, rather than in detail, in order to avoid obscuring the present invention.

The reader's attention is directed to all papers and documents which are filed concurrently with this specification and which are open to public inspection with this specification, and the contents of all such papers and documents are incorporated herein by reference. All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Furthermore, any element in a claim that does not explicitly state “means for” performing a specified function, or “step for” performing a specific function, is not to be interpreted as a “means” or “step” clause as specified in 35 U.S.C. Section 112(f). In particular, the use of “step of” or “act of” in the claims herein is not intended to invoke the provisions of 35 U.S.C. 112(f).

Before describing the invention in detail, first an introduction provides the reader with a general understanding of the present invention. Thereafter, specific details of various embodiment of the present invention are provided to give an understanding of the specific aspects.

(1) Introduction

The invention of the present disclosure is generally directed to the field of real world data and medical research data processing, acquisition and analysis (although it should be understood that the invention is not to be limited to such embodiments and should be afforded the widest scope possible). In one aspect, the invention relates to techniques for drawing upon a wide range of available real world data and medical research data for informing decisions related to further data processing, acquisition and analysis for the purposes of optimizing and accelerating the acceptance of cannabis-related medicine, increasing the development of cannabis-related medicine, increasing patient access to new cannabis-related medicine, increasing the diagnostics relating to cannabis-related medicine. Using the system herein, the system effectively provides a consistent data and research platform for cannabis ratios for specific conditions among humans and other animals.

(2) Specific Details of Various Embodiments

A system and technique is provided for enhancing performance of computer-assisted data operating algorithms in a medical research context. Datasets are compiled and accessed, which may include data from a wide range of resources. Non-limiting examples of such resources include controllable resources, such as real-world data collection methods, imaging systems, electrical systems, clinical laboratory systems, and pharmaco-kinetic systems. The datasets are analyzed by a human expert or a computer-based expert (such as an artificial intelligence (AI) system) and the algorithms are modified based upon feedback from the human expert or computer-based expert. Modifications may be made to a wide range of algorithms and based upon a wide range of data, such as available from an integrated knowledge base or real-world data. For example, modifications may be made in the algorithms providing enhanced functionality. Modifications may also be made on various bases, including patient-specific changes, population-specific changes, feature-specific changes, and so forth.

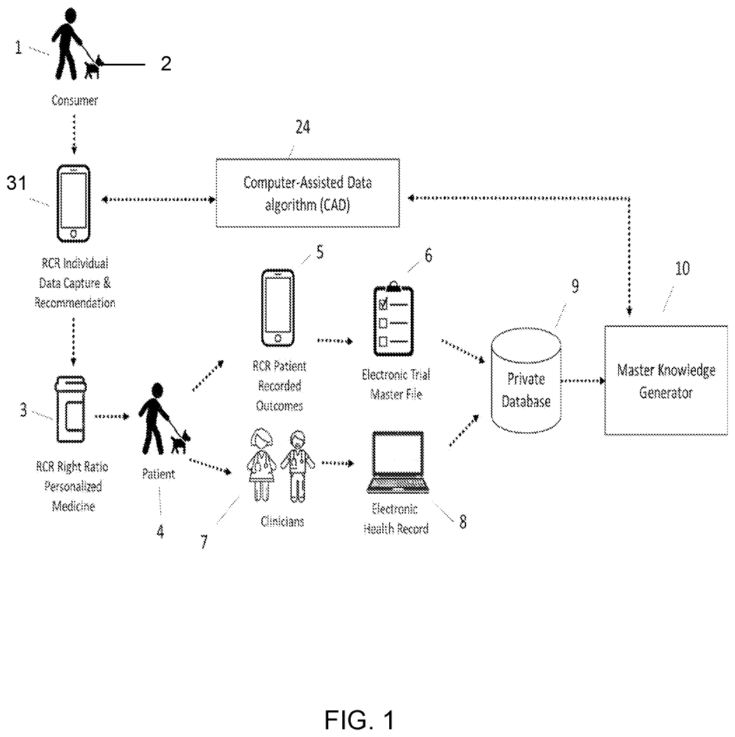

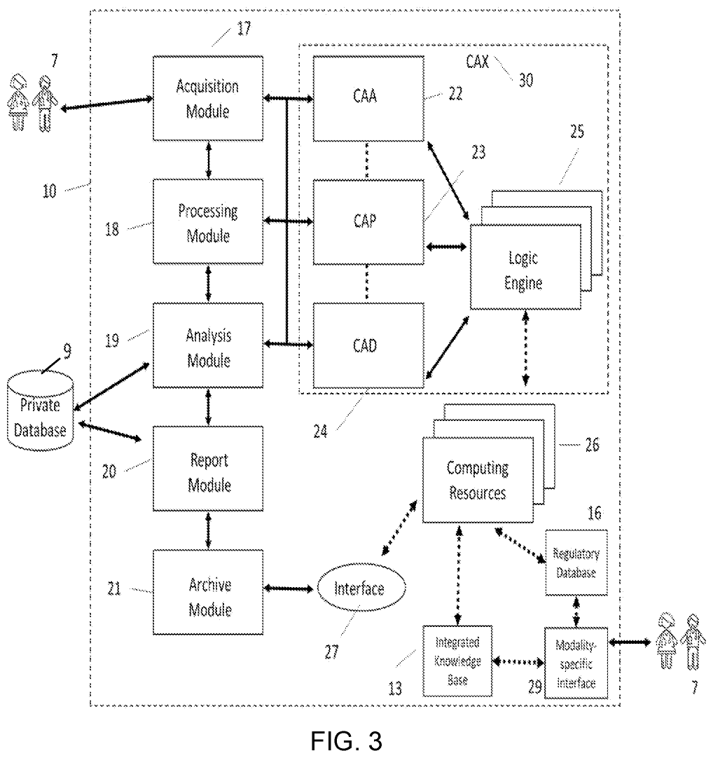

For further understanding, FIG. 1 provides a schematic diagram illustrating a data method and system that interfaces with consumers, patients, clinicians and researchers in accordance with an embodiment of the present invention. As shown in FIG. 1, a consumer 1 is a person who owns or cares for the wellbeing of a pet, sometimes referred to as a “pet parent”. The system is formed to allow the consumer 1 to input individual data relating to their pet 2 into a form or other system that allows for data input. As a non-limiting example, the system also includes a downloadable app or software that can be opened and operated by a user's computing device 31 (e.g., mobile phone, desktop computer, tablet computer, etc.). Thus, in operation, the consumer 1 opens the app on their computing device 31 and fills out pet-specific clinically validated measures and questionnaires designed to identify and evaluate the pet's breed, age, weight, medical concern, environmental factors, medical history and other pertinent information. Once the information is collected and submitted by the pet parent, the computer-assisted data operating algorithms (CAD) module 24 (as further described in FIG. 3) analyzes the pet data using the Master Knowledge Generator 10 and indicates to the pet parent (via their computing device 31), which formulation of the cannabinoid-terpenoid drug and doses are appropriate for their pet's condition, thereby creating a Right Ratio Personalized Medicine (RRPM) recommendation along with the appropriate safety information 3. As understood by those skilled in the art, the CAD module 24 and Master Knowledge Generator 10 are housed in an abstraction layer of the computing infrastructure that virtualizes and logically presents resources and services to users through application programming interfaces and API-enabled command-line or graphical interfaces.

Once entered into the Master Database Generator, the pet parent becomes part of a wellness system (further described herein) where they are sent alerts to administer medicine in order to increase patient compliance. The patient 4 is defined as a human or companion animal that has either already been entered into the Master Knowledge Generator 10 and been provided a Right Ratio Personalized Medicine recommendation 3 by means of completing the previously described data capture process (e.g., using a computing device 31), or is a human or a companion animal that is being monitored and data is being collected by a clinician, research team or other medically-related personnel 7.

The flow of information, as indicated by the arrows in FIG. 1, may include a wide range of types and vehicles for information exchange, as described more fully below. Interaction between the patient 4 may take any suitable form, typically depending upon the nature of the interface. In general, the patient 4 may interface with clinicians 7 through conventional clinical visits, as well as remotely by telephone, electronic mail, forms, and so forth. The patient 4 may also interact with elements of patient recorded outcomes 5 by either routine examinations designed to evaluate changes in specific medical conditions, adverse events, quality-of-life-changes, physical and emotional functioning, sleep, or via a range of data acquisition interfaces, which may include real world data forms, conventional patient history forms, systems for collecting and analyzing real world data, medical samples, body fluids, and so forth. Regular patient blood and pathology testing may also be required in certain circumstances and all data is captured, encrypted and entered into an Electronic Health Record (EHR) 8 or a FDA-compliant Electronic Trial Master File (ETMF) 6 that is then stored into an FDA-compliant database 9.

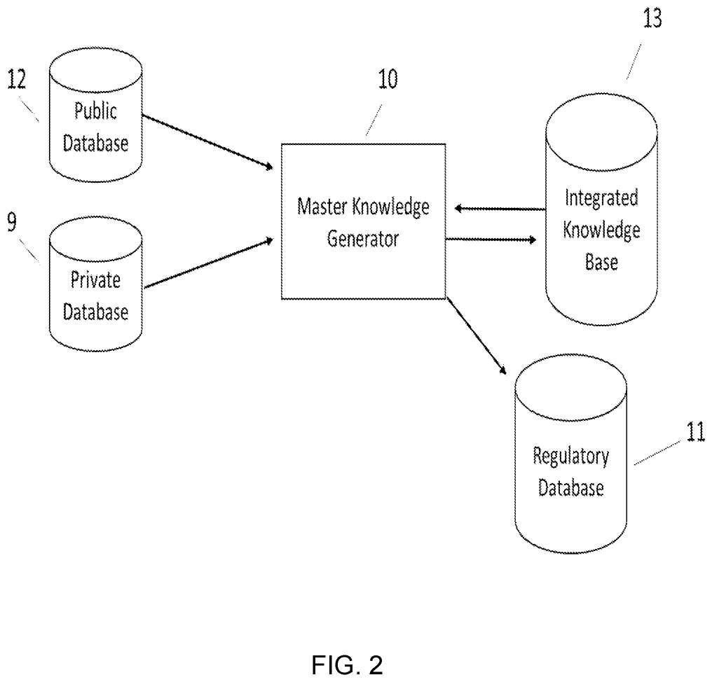

As noted generally in FIG. 1, the Master Knowledge Generator 10 and interconnection of the various resources, databases, and processing components can vary greatly. F or example, FIG. 1 illustrates a private database 9 as being linked to both the Master Knowledge Generator 10 and the Electronic Health Record 8 and an Electronic Trial Master File (ETMF) 6. Such arrangements will permit the private database, and the software contained therein, to extract and access information, while providing the information to the Master Knowledge Generator 10 upon demand. The Master Knowledge Generator 10, in certain instances, may directly extract or store information in the database where such information can be accessed and interpreted or translated. Similarly, the Master Knowledge Generator 10 can be linked to the Integrated Knowledge Base 13 (as described in FIG. 2). The Master Knowledge Generator 10, which may be subdivided into specific interface types or components, may thus be used to access knowledge directly from the Integrated Knowledge Base 13, or to command data be processed in a private database 9 to acquire, analyze, process or otherwise manipulate data. Such links between the data are illustrated diagrammatically in the figures for explanatory purposes. In specific systems, however, the high degree of integration may follow specific software modules or programs which perform specific analyses or correlations for specific patients, specific disease states, specific institutions, and so forth.

In FIG. 2, a private database 9, such as the database described in FIG. 1 used for collecting FDA-compliant patient data, or a public database 12 containing other relevant medical research information connects to the Master Knowledge Generator 10. The Master Knowledge Generator 10 has secure access controls, clear data audit trails and facilitates the management, security, compliance, analysis and reporting processes relating to data resources, such as private databases 9, public databases 12, the Integrated Knowledge Base 13 and others. In the present context, the Integrated Knowledge Base 13 is considered to include any and all types of available research data which can be processed by the Master Knowledge Generator 10 and made available to the clinician for providing the desired research. The Master Knowledge Generator 10 captures and monitors workflows and the resulting generated data in the various data resources.

The Master Knowledge Generator 10 and the data resources comply to a data governance framework that governs all use of data and is both “trial grade” and “regulator ready”. Master Knowledge Generator 10 and the data resources complete a de-identification process for clinical trials and for other regulatory needs, such as FDA approvals. The de-identification process covers the removal of all patient personal identifiable information (PII), is stored in separate regulatory database 11, and follows the guidance and protocols recommended in the FDA's 2018 report titled Use of Electronic Health Record Data in Clinical Investigations and the guidance and protocols recommended in the FDA's 2019 report titled Submitting Documents Using Real-World data and Real-World Evidence to FDA for Drugs and Biologics.

The Master Knowledge Generator 10, and the public database(s) 12 draw upon data from a range of data resources. The public database(s) 12 may be software-based and includes data access tools for drawing information from the various resources as described below or coordinating or translating the access of such information. In general, the public database(s) will unify raw data into a useable form. Any suitable form may be employed, and multiple forms may be employed, where desired, including hypertext markup language (HTML) extended markup language (XML), and so forth. In the present context, the Integrated Knowledge Base 13 is considered to include any and all types of available research data which can be processed by the Master Knowledge Generator 10 and made available for providing the desired research. In the simplest implementation, data may include a single source of data or more conventional data extraction techniques (e.g., questionnaires completed by a pet patient). However, the data may include many more and varied types of data. In general, data within the databases and Integrated Knowledge Base are digitized and stored to make the data available for extraction and analysis by the database(s) and the Master Knowledge Generator 10. Thus, even where more conventional data gathering resources are employed, the data is placed in a form which permits it to be identified and manipulated in the various types of analyses performed by the Master Knowledge Generator 10.

The Integrated Knowledge Base 13 is intended to include one or more repositories/resources of research or medical-related data in a broad sense. The Master Knowledge Generator 10 is intended as an interface and translator between the repositories/resources, including the Integrated Knowledge Base 13, and have the processing capabilities for carrying out desired operations on the data, including analysis, diagnosis, reporting, display, communication management directly to the patient and/or between the various clinicians and users, personalized medicine recommendations of formulation and dose (based on criteria such as, but not limited to disease or condition, the severity of that disease or condition, the age and weight and history of the patient), pharmacovigilance, new product development used for developing new drug formulations and/or new methods of treatment or application, clinical study design (such as defining the parameters by predicting which patients would respond best to which therapies, so that response rates could be maximized, and the probability of improved clinical outcomes could be increased), market research (such as defining or characterizing market preferences and depth), and other functions. The data itself may relate to patient-specific characteristics as well as to non-patient specific information, as for classes of persons, animals, species, systems and so forth. Moreover, the repositories/resources may include devoted systems for storing the data, or memory devices that are part of disparate systems. As noted above, the repositories/resources making up the Integrated Knowledge Base may be expandable and may be physically resident at any number of locations, typically linked by dedicated or open network links. Furthermore, the data contained in the Integrated Knowledge Base 13 may include both patient data (i.e., data relating specifically to a patient condition) and research data. Research data may include data representative of populations, species, segments, financial resources, physical resources (as at an institution or supplier), human resources, and so forth.

The medical researcher may interact with the Master Knowledge Generator 10 through conventional input devices, such as keyboards, computer mice, touch screens, portable or remote input and reporting devices. Moreover, the links between the Master Knowledge Generator 10, the Integrated Knowledge Base 13 and the public and private database(s) may include computer data exchange interconnections, network connections, local area networks, wide area networks, dedicated networks, virtual private network, and so forth.

As noted above and with respect to FIG. 3, the database resources may generally be thought of as information repositories which include means for acquiring medical research data through automated, semi-automated, or manual techniques in the Master Knowledge Generator 10. Such resources may be thought of as including certain general modules such as an acquisition module 17, a processing module 18, an analysis module 19, a report module 20, and an archive module 21. The nature of these various modules may differ widely, of course, depending upon the type of resource under consideration. Thus, the acquisition module 17 may include various types of electrical sensors, transducers, circuitry, imaging equipment, and so forth, used to acquire raw patient data. The acquisition module 17 may also include more real world data collection or data collection from human-based systems, such as questionnaires, surveys, forms, computerized and other input devices, and the like or may acquire data using software working with a wearable technology activity tracker allowing integration of tracker data with the reporting of information in the report module 20 used to monitor dosage and result, along with blood work, various data points such as breed, diagnosis, age, other medications, food and water intake, environmental conditions in order to optimize dosage among different people and different animal species, breeds and conditions or ailments. The report module 20 is configured to provide an alert where the patient 4 is taking medication which has a negative interaction with a new medication, supplement, or food or to assist and remind the patient when to take/administer the medication to in order to increase patient compliance which also provides a higher consistency of data collection. The report module 20 also sends out communication reminders informing the patient 4 when the optimal time to take the next dosage may be and initiates a request to the patient or clinician via the acquisition module 17 to complete a data acquisition process. The analysis module 19 also tracks improvement of symptoms and compares the information among a larger population to develop a minimum effective dose of a newly introduced medicine where, for example, that medicine has a dosage range and that medicine is cannabis-derived.

The nature and operation of the processing module 18, similarly will depend upon the nature of the acquisition module and of the overall resource type. Processing modules may thus include data conditioning, filtering, augmentation, and may also include such applications as spreadsheets, data compilation software, and the like. In electrical and imaging systems, the processing module may also include data enhancement circuits and software used to perform image and other types of data scaling, reconstruction, and display.

Analysis module 19 may include a wide range of applications which can be partially or fully automated. In electrical and imaging systems, for example, the analysis module may permit users to enhance or alter the display of data and reconstructed images. The analysis module may also permit some organization of clinician-collected data for evaluating the data or comparing the data to reference ranges, and the like. The report module 20 typically provides for an output or summary of the analysis performed by module 19. Reports may also provide an indication of techniques used to collect data, the number of data acquisition sequences performed, the types of sequences performed, patient conditions during such data acquisition, and so forth. Finally, the archive module 21 permits the raw, semi-processed, and processed data to be stored either locally at the acquisition system or- resource, or remote therefrom, such as in a database, repository, archiving system, and so forth.

The typical modules included within the controllable resources may be interfaced with programs (CAX) 30, to enhance the performance of various acquisition, processing and analysis functions. For example, various computer-assisted acquisition (CAA) routines 22 may be available for analyzing previous acquisition sequences, and for controlling or configuring subsequent data acquisition routines. Similarly, computer-assisted processing (CAP) modules 23 may interface with the processing module 18 to perform additional or enhance processing, depending upon previous processing and analysis of acquired data. Finally, programs such as computer-assisted data operating algorithms (CAD) modules 24 may be used to analyze received and processed data to provide some indication of possible findings that may be made from the data.

A non-limiting example of a specific CAA routine 22 includes data acquisition to ensure the data quality of real-world evidence through a wearable technology tracker (such as bracelets, mobile sensors, emotive headsets and smart devices), where the CAA is used for ensuring the completeness and accuracy of the required data as defined by the Acquisition Module 17 such as heartbeat, count of steps and geographic location has been acquired and is reasonable. If heart rate data was collected and count of steps and geographic location had not been collected, was partially collected, or the data collected seems to be an anomaly (such as high heart rate, high number of steps and no change in geographic location from previous data acquisition routine), the CAA will initiate a routine to collect new information from the Acquisition Module 17 until the data is deemed acquired, complete and accurate by the CAA 22. This ability of the invention ensures the “already in use” data requested by the Acquisition Module 17 is captured (and ultimately utilized) with a high degree of accuracy, can be monitored for inaccuracy,

A non-limiting example of enhanced processing by the CAP module 23 includes data append routines that involve adding new data elements to existing Private Database 9 to enrich already captured information and allows the components and data within the invention to be upgraded for capturing additional data through additional wearable technology trackers or for upgrading existing wearable technology trackers. A non-limiting scenario where this would occur, and the CAP module 23 would be utilized would be when adding an additional wearable technology tracker for collecting blood oxygen level and skin temperature and then appending the newly acquired data to the data record which has heart rate, number of steps and geographic location data. This enhanced processing by the CAP module 23 ensures more robust analysis in this example by increasing the data points from 3 to 5 variables, and increases the flexibility of the analysis by having the functionality to add or modify the analysis criteria as needed.

A non-limiting example of how the CAD module 24 works to analyze data and provide findings include using artificial intelligence routines to provide the ability to automate difficult cognitive tasks using “big data” for the purposes of learning abstract features from the raw data and augmenting decision making for the differential diagnosis of a medical condition and predicting the time to first treatment for patients diagnosed with a heart condition, or if there is inefficient data for the CAD module 24 to make a decision of reasonable certainty of the diagnosis, yet has a high enough certainty in the deep learning obtained to continue the research investigation, the CAD module 24 will initiate a routine with a program within the invention, CAX 30 that will initiate computer automated routines within the CAA 22 to request and acquire more recent data (such as heart rate, weight and age) from the patient 7, and initiate routines within the CAP 23 to append additional data (such as blood oxygen level and skin temperature) to enhance the research. In this scenario, both computer automated processes collectively enhance the artificial intelligence increasing the predictability of the CAD module 24 being able to diagnosis the heart condition with a higher degree of certainty and accuracy.

In the present disclosure regarding the Master Knowledge Generator 10 and the various types of controllable resource types and modalities, as well as of the modules used to aid in the acquisition, processing, analysis and predictive functions performed on the data from such resources, it should be noted that various links between these components and resources are available. Thus, in a typical application, a computer-assisted acquisition (CAA) module 22 may control or configure subsequent acquisition of data, such as video data, based upon the results of enhanced processing performed by a computer-assisted processing (CAP) module 23. A non-limiting example of how the CAA module 22 controls or configures acquisition of video data includes using a data acquisition routine via mobile crowd-sensing where video data is acquired using built-in sensors of a smart device such as a camera and a digital “chirp” used at a scheduled [pet-patient] feeding time where the crowd-sensing device captures a close up video of the [pet-patient's] pupils measuring the cognitive level of eye movement to determine cognitive responsiveness after the use of Right Ratio Personalized Medicine 3.

Similarly, such acquisition may result from output from a computer-assisted diagnosis (CAD) module 24, such as to refine potential recommendations made, based upon subsequent data acquisition 2. The CAD module 24 refines recommendations by applying artificial intelligence to a complex set of data from external sources. For example, using the same pupil video data captured previously discussed, the deep learning created via CAD 24 with the collective data in the internal Private Database 9 can assess and predict an estimated probability of a condition, and then apply this deep learning to the Master Knowledge Generator 10 which is then used to apply and compare the findings to other external sources such as a Public Database 12 researching the effectiveness of an external prescribed treatment or increasing the applied learnings by using and Integrated Knowledge Base 13 where deep learning is conducted across multiple conditions that correlations between different diseases are previously unknown.

In a similar manner, a computer-assisted processing (CAP) module 23 may command enhanced, different or subsequent processing by processing module 18 based upon output of computer-assisted (CAA) module 22 or of a computer-assisted diagnosis (CAD) module 24. For example, the CAP module 23 commands enhanced or different processing by processing routines that aggregates data and append probability scores of high correlation (conducted by the CAD 24 processing routine) between multiple, previously unknown factors such as disease types, canine genome vs human genome or treatment method vs treatment method. More specifically, the CAP module 23 processing routines are conducted across multiple databases such as a Private Database 9 that holds internal data to the research organization, Public Database 12 that houses and generates data from external 3^{rd} party research organizations that analyze the same research objectives as the internal research team, and Integrated Knowledge Bases 13 that research entirely different medical conditions for humans or animals. The various modules, both of the resources, and of the programs, then, permit a high degree of cyclic and interwoven data acquisition, processing and analysis by virtue of the integration of these modules into the overall system in accordance with the present techniques.

Also, for the typical controllable resource, the programs executed on the data, and used to provide enhanced acquisition, processing and analysis, may be driven by a logic engine 25 of the programs 22. As noted above, and as discussed in greater detail below, the logic engine 25 may incorporate a wide range of algorithms which link and integrate the output of programs, such as CAX algorithms, certain of which are noted as CAA, CAP and CAD modules 22, 23 and 24, and which prescribe or control subsequent acquisition, processing and analysis based upon programmed correlations, recommendations, and so forth. As also noted above, the programs (CAX) 30 are accessed by and implemented via the computing resources 26. The computing resources 26 may interface generally with the archive module 21 of the particular resource modality via an appropriate interface 27 as mentioned above. Finally, the computing resources 26 interface with the Integrated Knowledge Base 13 and Regulatory Database 11. It should be noted that the Integrated Knowledge Base 13 and Regulatory Database 11 may include modality-specific interfaces 29 which access repositories of information relating to the specific modality of the resource 13, 16, 18-21 and 26. Such modality-specific interfaces may include factors such as system settings, preferred settings for specific patients or populations, routines and protocols, data interpretation algorithms based upon the specific modality, and so forth.

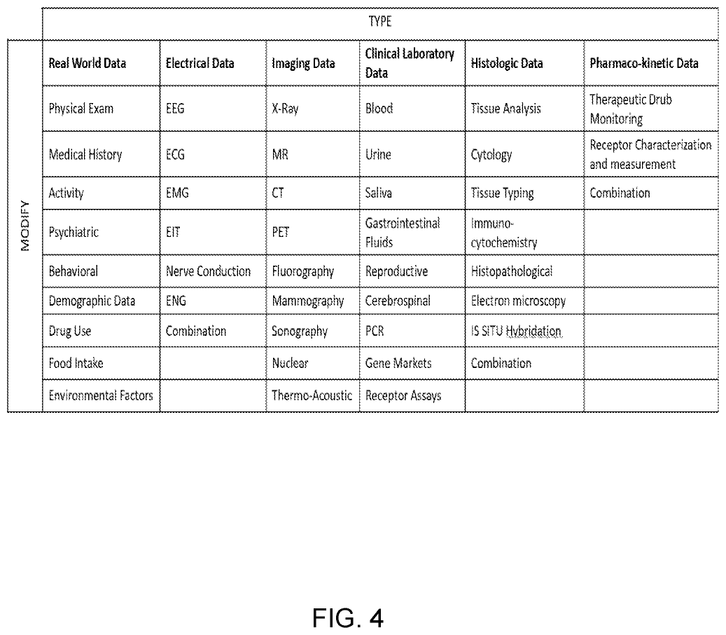

FIG. 4 is a table depicting various types of controllable resources, and the modalities of such resource types may include any available data resources which can be useful in performing the acquisition, processing, analysis functions offered by the present techniques. Specifically, the present technique contemplates that as few as a single resource may be provided, such as for integration of acquisition, processing and analysis over time, and, in a most useful configuration, a wide range of such resources are made available.

As noted above, such controllable resources may generally include real world data, electrical data sources, imaging data sources, clinical laboratory data sources, histologic data sources, pharmacokinetic data sources, and other miscellaneous sources of medical research data. While various reference data on each of these types and modalities may be included in the data resources, the types and modalities enumerated in the table are designed to acquire data which is patient-specific which is acquired either directly or indirectly from a patient or is trial-specific data following the necessary regulatory privacy and de-classification protocols, or the data is non-patient-specific and could include data from medical studies and research, general market and consumer data, geographic data referring to a particular population and so forth.

The invention is also a method for compiling data both before and after the medication is administered among medical doctors, including veterinarians, laboratories, patients and in the case of humans and mammals other than humans, pet owners, where a software working with a wearable technology activity tracker allowing integration of tracker data (e.g., heart rate, pulse, temperature, etc.) with the reporting of information used to monitor dosage and result, along with blood work, various data points such as breed, diagnosis, age, other medications, food and water intake, environmental conditions in order to optimize dosage among different people and different animal species, breeds and conditions or ailments. The software is configured to provide an alert where the wearer is taking medication which has a negative interaction with a new medication, supplement or food. The software also tracks improvement of symptoms and compares the information among a larger population to develop a minimum effective dose of a newly introduced medicine where, for example, that medicine has a dosage range and that medicine is cannabis—derived. The process also helps in conducting translational medicine, can be used to accelerate the FDA drug approval process, and can be used in generating clinical trial design by providing relevant and applicable clinical trial data.

As can be appreciated by those skilled in the art, the invention of the present disclosure includes any of the hardware/software, and any other component as may be necessary to implement the invention as described above. Thus, provided below are some example embodiments in which the system and method can be implemented. However, it should be noted that although specific implementations are provided below, the invention is not intended to be limited thereto as any suitable component/hardware/software, etc. can be used as understood by those skilled in the art to implement the present invention.

Thus, in various embodiments, the invention includes three “principal” aspects. The first is a system for medical research data, acquisition, and analysis. The system is typically in the form of a computer system operating software or in the form of a “hard-coded” instruction set. This system may be incorporated into a wide variety of devices that provide different functionalities. The second principal aspect is a method, typically in the form of software, operated using one or more networked data processing systems (computer) as implemented on any computing system, such as a computer, tablet computer, smart phone, etc. The third principal aspect is a computer program product. The computer program product generally represents computer-readable instructions stored on a non-transitory computer-readable medium such a hard drive, optical storage device, e.g., a compact disc (CD) or digital versatile disc (DVD), or a magnetic storage device such as a floppy disk or magnetic tape. Other, non-limiting examples of computer-readable media include hard disks, read-only memory (ROM), and flash-type memories. These aspects will be described in more detail below.

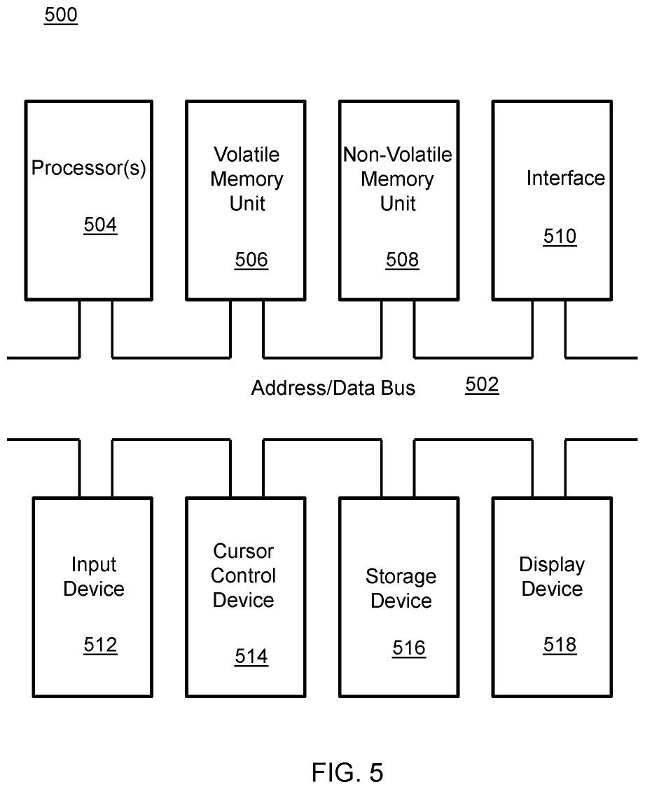

A block diagram depicting an example of a system (i.e., computer system 500) of the present invention is provided in FIG. 5. The computer system 500 is configured to perform calculations, processes, operations, and/or functions associated with a program or algorithm. In one aspect, certain processes and steps discussed herein are realized as a series of instructions (e.g., software program) that reside within computer readable memory units and are executed by one or more processors of the computer system 500. When executed, the instructions cause the computer system 500 to perform specific actions and exhibit specific behavior, such as described herein. In various aspects, the computer system 500 can be embodied in any device(s) that operates to perform the functions as described herein as applicable to the particular application, such as a desktop computer, a mobile or smart phone, a tablet computer, a computer embodied in a mobile platform, or any other device or devices that can individually and/or collectively execute the instructions to perform the related operations/processes as may be necessary to implement the invention as described herein.

The computer system 500 may include an address/data bus 502 that is configured to communicate information. Additionally, one or more data processing units, such as a processor 504 (or processors), are coupled with the address/data bus 502. The processor 504 is configured to process information and instructions. In an aspect, the processor 504 is a microprocessor. Alternatively, the processor 504 may be a different type of processor such as a parallel processor, application-specific integrated circuit (ASIC), programmable logic array (PLA), complex programmable logic device (CPLD), or a field programmable gate array (FPGA) or any other processing component operable for performing the relevant operations.

The computer system 500 is configured to utilize one or more data storage units. The computer system 500 may include a volatile memory unit 506 (e.g., random access memory (“RAM”), static RAM, dynamic RAM, etc.) coupled with the address/data bus 502, wherein a volatile memory unit 506 is configured to store information and instructions for the processor 504. The computer system 500 further may include a non-volatile memory unit 508 (e.g., read-only memory (“ROM”), programmable ROM (“PROM”), erasable programmable ROM (“EPROM”), electrically erasable programmable ROM “EEPROM”), flash memory, etc.) coupled with the address/data bus 502, wherein the non-volatile memory unit 508 is configured to store static information and instructions for the processor 504. Alternatively, the computer system 500 may execute instructions retrieved from an online data storage unit such as in “Cloud” computing. In an aspect, the computer system 500 also may include one or more interfaces, such as an interface 510, coupled with the address/data bus 502. The one or more interfaces are configured to enable the computer system 500 to interface with other electronic devices and computer systems, including other tablets, phones, or other items as may be applicable to implementing the invention as described herein. The communication interfaces implemented by the one or more interfaces may include wireline (e.g., serial cables, modems, network adaptors, etc.) and/or wireless (e.g., wireless modems, wireless network adaptors, etc.) communication technology. Further, one or more processors 504 can be associated with one or more associated memories, where each associated memory is a non-transitory computer-readable medium. Each associated memory can be associated with a single processor 504 (or device), or a network of interacting processors 504 (or devices), such as a network of devices (e.g., individual computers/tablets/phones, etc. as used by users to upload or otherwise implement the invention as described herein)

In one aspect, the computer system 500 may include an input device 512 coupled with the address/data bus 502, wherein the input device 512 is configured to communicate information and command selections to the processor 504. In accordance with one aspect, the input device 512 is an alphanumeric input device, such as a keyboard, that may include alphanumeric and/or function keys. Alternatively, the input device 512 may be an input device other than an alphanumeric input device. In an aspect, the computer system 500 may include a cursor control device 514 coupled with the address/data bus 502, wherein the cursor control device 514 is configured to communicate user input information and/or command selections to the processor 104. In an aspect, the cursor control device 514 is implemented using a device such as a mouse, a track-ball, a track-pad, an optical tracking device, or a touch screen. The foregoing notwithstanding, in an aspect, the cursor control device 514 is directed and/or activated via input from the input device 512, such as in response to the use of special keys and key sequence commands associated with the input device 512. In an alternative aspect, the cursor control device 514 is configured to be directed or guided by voice commands.

In an aspect, the computer system 500 further may include one or more optional computer usable data storage devices, such as a storage device 516, coupled with the address/data bus 502. The storage device 516 is configured to store information and/or computer executable instructions. In one aspect, the storage device 516 is a storage device such as a magnetic or optical disk drive (e.g., hard disk drive (“HDD”), floppy diskette, compact disk read only memory (“CD-ROM”), digital versatile disk (“DVD”)). Pursuant to one aspect, a display device 518 is coupled with the address/data bus 502, wherein the display device 518 is configured to display video and/or graphics. In an aspect, the display device 518 may include a cathode ray tube (“CRT”), liquid crystal display (“LCD”), field emission display (“FED”), plasma display, touch screen display on a mobile phone, tablet, or computer, or any other display device suitable for displaying video and/or graphic images and alphanumeric characters recognizable to a user.

The computer system 500 presented herein is an example computing environment in accordance with an aspect. However, the non-limiting example of the computer system 500 is not strictly limited to being a computer system. For example, an aspect provides that the computer system 500 represents a type of data processing analysis that may be used in accordance with various aspects described herein. Moreover, other computing systems may also be implemented. Indeed, the spirit and scope of the present technology is not limited to any single data processing environment. Thus, in an aspect, one or more operations of various aspects of the present technology are controlled or implemented using computer-executable instructions, such as program modules, being executed by a computer. In one implementation, such program modules include routines, programs, objects, components and/or data structures that are configured to perform particular tasks or implement particular abstract data types. In addition, an aspect provides that one or more aspects of the present technology are implemented by utilizing one or more distributed computing environments, such as where tasks are performed by remote processing devices that are linked through a communications network, or such as where various program modules are located in both local and remote computer-storage media including memory-storage devices.

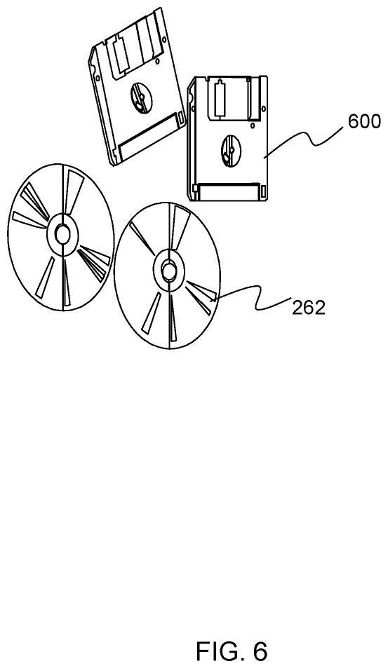

An illustrative diagram of a computer program product (i.e., storage device) embodying the present invention is depicted in FIG. 6. The computer program product is depicted as floppy disk 600 or an optical disk 602 such as a CD or DVD. However, as mentioned previously, the computer program product generally represents computer-readable instructions stored on any compatible non-transitory computer-readable medium. The instructions can be transmitted and or downloaded for use by individual user on their individual device or otherwise stored on a platform for operation by an associated processor. The term “instructions” as used with respect to this invention generally indicates a set of operations to be performed on a computer, and may represent pieces of a whole program or individual, separable, software modules. Non-limiting examples of “instruction” include computer program code (source or object code) and “hard-coded” electronics (i.e. computer operations coded into a computer chip). The “instruction” is stored on any non-transitory computer-readable medium, such as in the memory of a computer or on a floppy disk, a CD-ROM, and a flash drive, or in the hard drive of a smart phone, tablet computer, etc. In either event, the instructions are encoded on a device as non-transitory computer-readable medium.

Finally, while this invention has been described in terms of several embodiments, one of ordinary skill in the art will readily recognize that the invention may have other applications in other environments. It should be noted that many embodiments and implementations are possible. Further, the following claims are in no way intended to limit the scope of the present invention to the specific embodiments described above. In addition, any recitation of “means for” is intended to evoke a means-plus-function reading of an element and a claim, whereas, any elements that do not specifically use the recitation “means for”, are not intended to be read as means-plus-function elements, even if the claim otherwise includes the word “means”. Further, while particular method steps have been recited in a particular order, the method steps may occur in any desired order and fall within the scope of the present invention.

What is claimed is:

1. A system for medical research data processing, acquisition and analysis, comprising:
a master knowledge generator, the master knowledge generator being operable for performing operations of:
receiving, from a private database, patient specific data for a patient;
receiving medicine information from an integrated knowledge base;
generating a right ratio personalized medicine recommendation for the patient based on the patient specific data and the medicine information from the integrated knowledge base;
receiving tracker data on the patient while the patient is taking the personalized medicine recommendation; and
updating the right ratio personalized medicine recommendation for the patient based on the tracker data.

2. The system as set forth in claim 1, wherein the master knowledge generator checks the personalized medicine recommendation against a regulatory database to ensure regulatory compliance.

3. The system as set forth in claim 2, further comprising a computer-assisted acquisition (CAA) module, the CAA module operable for performing operations of:
receiving parameters detailing a resource type used to acquire data;
receiving parameters detailing a data type and condition of data to acquire;
receiving medical information from a device to capture the data;
transferring the data that has been captured to a processing module that allocates the data to a computer program for analysis;
reporting on the data obtained using a report module; and
archiving the data using an archive module.

4. The system as set forth in claim 4, further comprising a computer-assisted processing (CAP) module, the CAP module operable for performing operations of:
receiving parameters detailing computer resources needed to access additional data;
receiving parameters detailing a source and data type to acquire the additional data;
receiving parameters detailing conditions to acquire the additional data;
updating a patient data record in the private database with the additional data;
transferring the additional data to other computer processing modules for analysis and storage;
reporting on the data using the report module; and
archiving the data using the archive module.

5. The system as set forth in claim 5, further comprising a computer-assisted data operating algorithms (CAD) module, the CAD module operable for performing operations of:
receiving parameters, from a computer-assisted program, detailing the data, an analysis of the data, a type of the data, a source of the data and logic needed to conduct artificial intelligence/deep learning routines that extract features from the data and augment decision making for medical purposes;
receiving parameters detailing the conditions to initiate the artificial intelligence/deep learning routines;
updating a patient data record with the logic needed to conduct artificial intelligence/deep learning routines;
updating one or more other databases with artificial intelligence/deep learning routines;
updating the artificial intelligence/deep learning routines to other computer processing modules for additional analysis and storage;
reporting on the additional analysis and artificial intelligence/deep learning using the report module; and
archiving the analysis and artificial intelligence/deep learning.

6. The system as set forth in claim 1, further comprising a computer-assisted acquisition (CAA) module, the CAA module operable for performing operations of:
receiving parameters detailing a resource type used to acquire data;
receiving parameters detailing a data type and condition of data to acquire;
receiving medical information from a device to capture the data;
transferring the data that has been captured to a processing module that allocates the data to a computer program for analysis;
reporting on the data obtained using a report module; and
archiving the data using an archive module.

7. The system as set forth in claim 1, further comprising a computer-assisted processing (CAP) module, the CAP module operable for performing operations of:
receiving parameters detailing computer resources needed to access additional data;
receiving parameters detailing a source and data type to acquire the additional data;
receiving parameters detailing conditions to acquire the additional data;
updating a patient data record in the private database with the additional data;
transferring the additional data to other computer processing modules for analysis and storage;
reporting on the data using a report module; and
archiving the data using a archive module.

8. The system as set forth in claim 1, further comprising a computer-assisted data operating algorithms (CAD) module, the CAD module operable for performing operations of:
receiving parameters, from a computer-assisted program, detailing the data, an analysis of the data, a type of the data, a source of the data and logic needed to conduct artificial intelligence/deep learning routines that extract features from the data and augment decision making for medical purposes;
receiving parameters detailing the conditions to initiate the artificial intelligence/deep learning routines;
updating a patient data record with the logic needed to conduct artificial intelligence/deep learning routines;
updating one or more other databases with artificial intelligence/deep learning routines;
updating the artificial intelligence/deep learning routines to other computer processing modules for additional analysis and storage;
reporting on the additional analysis and artificial intelligence/deep learning using a report module; and
archiving the analysis and artificial intelligence/deep learning.

9. A computer program product for medical research data processing, acquisition and analysis, comprising:
a non-transitory computer-readable medium having executable instructions encoded thereon, such that upon execution of the instructions by one or more processors, the one or more processors perform operations of:
receiving, from a private database, patient specific data for a patient;
receiving medicine information from an integrated knowledge base;
generating a right ratio personalized medicine recommendation for the patient based on the patient specific data and the medicine information from the integrated knowledge base;
receiving tracker data on the patient while the patient is taking the personalized medicine recommendation; and
updating the right ratio personalized medicine recommendation for the patient based on the tracker data.

10. The computer program product as set forth in claim 9, further comprising instructions for causing the one or more processors to perform an operation of checking the personalized medicine recommendation against a regulatory database to ensure regulatory compliance.

11. The computer program product as set forth in claim 9, further comprising instructions for causing the one or more processors to perform operations of:
receiving parameters detailing a resource type used to acquire data;
receiving parameters detailing a data type and condition of data to acquire;
receiving medical information from a device to capture the data;
transferring the data that has been captured to a processing module that allocates the data to a computer program for analysis;
reporting on the data obtained using a report module; and
archiving the data using an archive module.

12. The computer program product as set forth in claim 9, further comprising instructions for causing the one or more processors to perform operations of:
receiving parameters detailing computer resources needed to access additional data;
receiving parameters detailing a source and data type to acquire the additional data;
receiving parameters detailing conditions to acquire the additional data;
updating a patient data record in the private database with the additional data;
transferring the additional data to other computer processing modules for analysis and storage;
reporting on the data using a report module; and
archiving the data using an archive module.

13. The computer program product as set forth in claim 9, further comprising instructions for causing the one or more processors to perform operations of:
receiving parameters, from a computer-assisted program, detailing the data, an analysis of the data, a type of the data, a source of the data and logic needed to conduct artificial intelligence/deep learning routines that extract features from the data and augment decision making for medical purposes;
receiving parameters detailing the conditions to initiate the artificial intelligence/deep learning routines;
updating a patient data record with the logic needed to conduct artificial intelligence/deep learning routines;
updating one or more other databases with artificial intelligence/deep learning routines;
updating the artificial intelligence/deep learning routines to other computer processing modules for additional analysis and storage;
reporting on the additional analysis and artificial intelligence/deep learning using a report module; and
archiving the analysis and artificial intelligence/deep learning.

14. A computer implemented method for medical research data processing, acquisition and analysis, comprising an act of:
causing one or more processers to execute instructions encoded on a non-transitory computer-readable medium, such that upon execution, the one or more processors perform operations of:
receiving, from a private database, patient specific data for a patient;
receiving medicine information from an integrated knowledge base;
generating a right ratio personalized medicine recommendation for the patient based on the patient specific data and the medicine information from the integrated knowledge base;
receiving tracker data on the patient while the patient is taking the personalized medicine recommendation; and
updating the right ratio personalized medicine recommendation for the patient based on the tracker data.

15. The computer implemented method as set forth in claim 15, further comprising an act of causing the one or more processors to perform an operation of checking the personalized medicine recommendation against a regulatory database to ensure regulatory compliance.

16. The computer implemented method as set forth in claim 15, further comprising an act of causing the one or more processors to perform operations of:
receiving parameters detailing a resource type used to acquire data;
receiving parameters detailing a data type and condition of data to acquire;
receiving medical information from a device to capture the data;
transferring the data that has been captured to a processing module that allocates the data to a computer program for analysis;
reporting on the data obtained using a report module; and
archiving the data using an archive module.

17. The computer implemented method as set forth in claim 15, further comprising an act of causing the one or more processors to perform operations of:
receiving parameters detailing computer resources needed to access additional data;
receiving parameters detailing a source and data type to acquire the additional data;
receiving parameters detailing conditions to acquire the additional data;
updating a patient data record in the private database with the additional data;
transferring the additional data to other computer processing modules for analysis and storage;
reporting on the data using a report module; and
archiving the data using an archive module.

18. The computer implemented method as set forth in claim 15, further comprising an act of causing the one or more processors to perform operations of:
receiving parameters, from a computer-assisted program, detailing the data, an analysis of the data, a type of the data, a source of the data and logic needed to conduct artificial intelligence/deep learning routines that extract features from the data and augment decision making for medical purposes;
receiving parameters detailing the conditions to initiate the artificial intelligence/deep learning routines;
updating a patient data record with the logic needed to conduct artificial intelligence/deep learning routines;
updating one or more other databases with artificial intelligence/deep learning routines;
updating the artificial intelligence/deep learning routines to other computer processing modules for additional analysis and storage;
reporting on the additional analysis and artificial intelligence/deep learning using a report module; and
archiving the analysis and artificial intelligence/deep learning.
